(19) United States
(12) Patent Application Publication
(10) Pub. No.: US 2008/0172032 A1
(43) Pub. Date: Jul. 17, 2008

(54) Method for preventing tissue damage associated with irrigation of tissue with an antimicrobial solution

(76) Inventors: James Pitzer Gills, Palm Harbor, FL (US); James Pitzer Gills, Clearwater Beach, FL (US)

Correspondence Address: KRAMER & AMADO, P.C., 1725 DUKE STREET, SUITE 240, ALEXANDRIA, VA, 22314, US

(21) Appl. No.: 11/652,102

(22) Filed: Jan. 11, 2007

(51) Int. Cl. A61M 37/00

(52) U.S. Cl. 604/500

(57) ABSTRACT

Methods for preventing tissue damage associated with irrigation of biological tissue with a povidone-iodine solution by irrigating the tissue to be treated with povidone-iodine solutions characterized by a pH of about 6.8 to about 8.0.

### BACKGROUND

Povidone-iodine solutions are used in the medical arts as non-antibiotic antiseptics. The solutions are used both as topical antiseptics and as irrigating antiseptics to be applied prior to and/or following surgery. The surgical applications of povidone-iodine solutions are recommended throughout a diverse range of surgical procedures due to the antiseptic properties of the solutions against a wide spectrum of microbial fauna and the low allergic risk associated with their use. However, povidone-iodine solutions have been linked with tissue damage to the irrigated tissue as well as decreased wound healing. It has been hypothesized that the use of iodine as an antiseptic agent attacks the enzymes of mammalian cells followed by an attack on the cell membranes.

Various studies have documented the possible tissue damage caused by the application of povidone-iodine solutions. When applied to rabbit knee articular and periatricular connective tissue, povidone-iodine scrub solution was associated with severe gross and histologic damage to articular cartilage, synovia and muscle. In contrast, povidone-iodine prep solution injected directly into the rabbit knee caused minimal gross and histological damage. Other studies have shown that tissue damage caused by povidone-iodine solutions may be concentration specific, where lower concentrations of povidone-iodine solutions have a decreased risk of tissue damage. When applied to rabbit eyes, povidone-iodine solutions induced significant endothelial damage at concentrations of 0.1%. At concentrations of 1%, povidone-iodine solutions caused comeal edema and endothelial cell loss as demonstrated by pachymetry, histopathology, and elevated intraocular pressure.

Despite the possible tissue damaged caused by povidone-iodine solutions, the antiseptic benefits of such solutions promotes their continued use. In particular, povidone-iodine solutions have made a significant contribution to pre- and post-operative ocular surgical prophylaxis, ophthalmia neonatorum prophylaxis and treatment of bacterial conjunctivitis. Varying concentrations of povidone-iodine solutions have been used in the aforementioned methods including 5% solution for before ocular surgery, 1.25% solution after ocular surgery, 2.5% solution at birth and 1.25% solution for some forms of conjunctivitis.

Of particular importance is the use of povidone-iodine solutions in pre- and post-operative surgical prophylaxis. However, the use of such solutions for pre- and post-operative prophylactic irrigation has resulted in damage to the sensitive tissues of the eye. The tissue damage may present as extreme eye dryness and painful irritation, often arising after surgery. A specific condition characterized by eye dryness is known as Tear Deficiency Syndrome. The diagnosis of a Tear Deficiency Syndrome is made formally by the Schirmer Test. A standard filter strip is hinged in the lateral conjunctival cul-de-sac and the extent of wetting of the strip is measured after five minutes. Less than 3 mm of wetting is considered diagnostic of tear deficiency. Post-operative Tear Deficiency Syndrome is currently treated using temporary therapies such as artificial tears. However, there is no current preventative method for the long term elimination of post-operative Tear Deficiency Syndrome.

### SUMMARY

In light of the present need for a method preventing tissue damage associated with irrigation of biological tissue with povidone-iodine solutions, a brief summary of various exemplary embodiments is presented. Some simplifications and omission may be made in the following summary, which is intended to highlight and introduce some aspects of the various exemplary embodiments, but not to limit the scope of the methods. Detailed descriptions of various exemplary embodiments adequate to allow those of ordinary skill in the art to make and use the methods follow in later sections.

Various exemplary embodiments relate to a method for preventing tissue damage associated with irrigation of biological tissue with an antimicrobial solution by irrigating biological tissue with a povidone-iodine solution characterized by a pH of about 6.8-8.0.

In various exemplary embodiments, the biological tissue is irrigated in preparation for surgery or following surgery. Exemplary embodiments of tissues to be irrigated include, but are not limited to, the following: ophthalmic tissue, mucosal tissue, intestinal tissue, otic tissue, nasal tissue, oral tissue, esophageal tissue, musculoskeletal tissue and/or gynecological tissue. Some exemplary embodiments include methods for treating ocular conjunctivitis caused by bacterial, fungal and/or viral infection.

Two particular types of methods for preventing tissue damage, especially tissue damage associated with irrigation of ophthalmic tissue include methods of preventing endothelial toxicity and methods for preventing tear deficiency syndrome.

### DETAILED DESCRIPTION

For simplicity and illustrative purposes, the principles are shown by way of examples of compositions and methods described. In the following description, numerous specific details are set forth in order to provide a thorough understanding of the examples. It will be apparent however, to one of ordinary skill in the art, that the examples may be practiced without limitation to these specific details. In other instances, well known methods and compositions are not described in detail so as not to unnecessarily obscure understanding of the examples.

In various exemplary embodiments, povidone-iodine solutions are used as antiseptic solutions in pre- and post-operative procedures. The terms pre- and post-operative encompass procedures taking place prior to or following an operation or surgery. The povidone-solutions are directly applied to the biological tissue on which the operation or surgery is performed. Particular of the types of biological tissue to be treated include mammalian tissue. In various exemplary embodiments, the mammalian tissue to be treated includes, but is not limited to ophthalmic tissue, endothelial tissue, mucosal tissue, intestinal tissue, otic tissue, nasal tissue, oral tissue, esophageal tissue, muscular tissue, connective tissue, skeletal tissue, epidermal tissue and/or gynecological tissue. Muscular, connective and skeletal tissue may be grouped together under the term musculoskeletal tissue. The term gynecological tissue defines a wide range of tissues or organs associated with the female reproductive system, including but not limited to vaginal and/or uterine tissue.

Some exemplary methods for preventing tissue damage associated with application of antimicrobial solutions include treating ocular conjunctivitis with povidone-iodine solutions. The ocular conjunctivitis may be caused by bacterial, fungal and/or viral infection.

In various exemplary embodiments, the povidone-iodine solutions are neutralized prior to, being applied to the biological tissue. The pH of such solutions is preferably neutralized to having a pH between 6.8 and 8.5. In various exemplary embodiments, the povidone-iodine solutions have a pH of in the range of about 7.0 and 8.0. The term about, as used consistently throughout the specification, means a variation of plus or minus 10%. The method of applying neutralized povidone-iodine solutions is useful for preventing tissue damage when used in all biological tissue types which are able to withstand conventional povidone-iodine solutions without tissue damage.

The method may further include the use of diluted povidone-iodine solutions. In various embodiments, the povidone-iodine solutions may be diluted from about 1:2 to about 1:1000. Particular exemplary embodiments include dilutions from about 1:10 to 1:500. The particular dilutions when used in the methods for preventing tissue damage may vary according to the type of biological tissue to be treated and the operation or surgical procedure that accompanies the irrigation with antiseptic solutions. In various exemplary embodiments, a higher dilution (i.e. lower concentration) is used for prophylaxis, whereas a lower dilution (i.e. higher concentration) or even an undiluted solution is used for a frank infection or where asepsis is of particular concern.

Conventional povidone-iodine solutions are commercially available for use in antiseptic irrigations. Such conventional solutions are not neutralized. A brand name of convention povidone-iodine solutions is the brand BETADINE® available from Purdue Pharma. BETADINE® Solution is described as 10% povidone-iodine in an aqueous solution having 1% available iodine. The methods for preventing tissue damage associated with irrigation of biological tissue include neutralizing commercially available povidone-iodine solutions.

In various exemplary embodiments, neutralized povidone-iodine solutions are neutralized with one or more physiologically-tolerated, non-toxic alkaline compounds. Various examples of such compounds include, but are not limited to, sodium hydroxide, potassium hydroxide, sodium bicarbonate, disodium phosphonate and mixtures thereof. In various embodiments the neutralized solutions are mixed with aqueous salt solutions. Commercially available salt solutions include BSS®, available from Alcon Laboratories, Inc. BSS® Sterile Irrigating Solution is a sterile physiological balanced salt solution, each mL containing Sodium Chloride (NaCl) 0.64%, Potassium Chloride (KCl) 0.075%, Calcium Chloride Dihydrate (CaCl₂.2H₂O) 0.048%, Magnesium Chloride Hexahydrate (MgCl₂.6H₂O) 0.03%, Sodium Acetate Trihydrate (C₂H₃NaO₂.3H₂O) 0.39%, Sodium Citrate Dihydrate (C₆H₅Na₃O₇.2H₂O) 0.17%, Sodium Hydroxide and/or Hydrochloric Acid (to adjust pH), and Water.

An exemplary embodiment of a neutralized povidone-iodine solution includes 10 cc povidone-iodine (10% in aqueous solution); 7 cc sodium bicarbonate and 3 cc BSS®. In various exemplary embodiments, the concentration of povidone-iodine solution ranges from about 0.5% to about 5% povidone-iodine.

In various exemplary embodiments, a method for preventing tissue damage associated with the irrigation of biological tissue with an antimicrobial solution includes irrigating ophthalmic tissue with a povidone-iodine solution characterized by a pH of about 6.8-8.0. The ophthalmic tissue is irrigated with the neutralized povidone-iodine solution prior to or following ophthalmic surgery.

The term ophthalmic surgery includes, but is not limited, to any surgery on or around the eye, including, but not limited to cataract surgery, glaucoma surgery, retina surgery, and corrective vision surgery, ICL, CLEAR, and LASIK. In various exemplary embodiments, the purpose of the irrigation is to provide asepsis prior to intraocular or extraocular procedures. In various exemplary embodiments, the method of irrigating ophthalmic tissue with neutralized povidone-iodine solutions prevents tissue damage, particularly toxicity. In various embodiments, the method of irrigating ophthalmic tissue with neutralized povidone-iodine solutions prevents tissue damage which presents as tear deficiency syndrome.

During an exemplary survey of surgical patients from a clinical practice, use of conventional povidone-iodine solutions resulted in an over 40% incidence of tear deficiency syndrome in post-operative cataract patients. Using the method for preventing tissue damage by pre-and/or post-operative irrigation with a povidone-iodine characterized by a pH of about 6.8-8.0 eliminated the incidence of post-operative tear deficiency syndrome in all cataract patients.

What is claimed is:

1. A method for preventing tissue damage associated with irrigation of biological tissue with an antimicrobial solution, comprising:
irrigating biological tissue to be irrigated with an antimicrobial irrigation solution;
wherein the antimicrobial irrigation solution comprises a povidone-iodine solution; and
wherein the povidone-iodine solution is characterized by a pH of about 6.8-8.0.

2. The method of claim 1 wherein the biological tissue is irrigated in preparation for surgery.

3. The method of claim 1, wherein the biological tissue is irrigated after surgery.

4. The method of claim 1, wherein the biological tissue to be irrigated is ophthalmic tissue.

5. The method of claim 4, wherein the ophthalmic tissue is irrigated before or after ophthalmic surgery is performed on the tissue.

6. The method of claim 5, further comprising preventing endothelial toxicity.

7. The method of claim 5, further comprising preventing Tear Deficiency Syndrome.

8. The method of claim 1, wherein the biological tissue is mucosal tissue.

9. The method of claim 1, wherein the biological tissue is intestinal tissue.

10. The method of claim 1, wherein the biological tissue is otic tissue.

11. The method of claim 1, wherein the biological tissue is nasal tissue.

12. The method of claim 1, wherein the biological tissue is oral tissue.

13. The method of claim 1, wherein the biological tissue is esophageal tissue.

14. The method of claim 1, wherein the biological tissue is musculoskeletal tissue.

15. The method of claim 1, wherein the biological tissue is gynecological tissue.

16. A method for preventing endothelial toxicity associated with irrigation of biological tissue with an antimicrobial solution, comprising:
irrigating endothelial tissue to be irrigated with an antimicrobial irrigation solution;
wherein the antimicrobial irrigation solution comprises a povidone-iodine solution; and
wherein the povidone-iodine solution is characterized by a pH of about 6.8-8.0.

17. The method of claim 16, wherein the endothelial tissue is irrigated prior to surgery.

18. The method of claim 17, wherein the endothelial tissue is ophthalmic tissue.

19. A method for preventing post-operative Tear Deficiency Syndrome, comprising:
irrigating ophthalmic tissue to be irrigated with an antimicrobial irrigation solution prior to or following ophthalmic surgery on a patient;
wherein the antimicrobial irrigation solution comprises a povidone-iodine solution;
wherein the povidone-iodine solution is characterized by a pH of about 6.8-8.0; and
wherein the patient does not suffer from Tear Deficiency Syndrome following the ophthalmic surgery.

20. The method of claim 19, wherein the ophthalmic surgery is cataract surgery.
